(19) United States
(12) Patent Application Publication
(10) Pub. No.: US 2026/0256421 A1
(43) Pub. Date: Sep. 3, 2026

(54) APPARATUS AND METHOD FOR THE ASSESSMENT OF PAIN

(71) Applicant: Yaqi Xie, London (GB)

(72) Inventor: Yaqi Xie, London (GB)

(21) Appl. No.: 18/873,463

(22) PCT Filed: Jun. 9, 2023
(86) PCT No.: PCT/EP2023/065510

§ 371 Date: Dec. 10, 2024

(30) Foreign Application Priority Data
Jun. 10, 2022 (GB) 2208551.8
Apr. 21, 2023 (GB) 2305887.8

(51) Int. Cl. A61B 5/00; A61B 5/22

(52) U.S. Cl. CPC A61B 5/4827; A61B 5/225; A61B 2562/0247

(57) ABSTRACT

A device comprising pressure sensors that measure the pressure of an applied touch; the duration of the touch; and enables an assessment of pain including the nature of the pain and the magnitude of the pain, the preferred device can be held in the human hand comprising a first section of a stretchable material of known stiffness whereby a user can transfer their pain tangibly by exerting the associated pressure onto the section.

### FIELD

The present invention relates to an apparatus and method for the assessment of pain in living species particularly human beings and particularly assesses pain through touch. The apparatus and method may be used to capture and measure pain in real time as it occurs.

### BACKGROUND

The assessment of pain is important in medical and caring activities both in determining a condition but also in determining the appropriate treatment for the source of the pain. This invention relies on touch pressure and touch behaviour during pain and uses analytics such as AI analytics to provide a pain score which can be used to provide treatments and/or pain predictions. In a preferred embodiment the invention can enable patients to visualise their pain and furthermore can be calibrated for each user.

Existing pain assessment techniques, such as the completion of pain assessment charts that rely on various pain scales with attributed numerical values or verbal/visual pain scales, provide an indication of the nature and degree of the pain. However, these methods are highly subjective and rely on a pain experience in memory, making it difficult to capture pain in real-time. Consequently, the current pain assessment tool may not provide an accurate assessment of pain, and the results can be highly variable. Furthermore, the assessment chart does not provide a comprehensive picture of the development of pain over time. Additionally, these techniques to not lend themselves to capturing pain in real time as it occurs.

United States Patent Application Publication number 2022/0104760 seeks to overcome some of these difficulties and provides a method of determining a representation of pain experienced by an individual by capturing information concerning at least two visible features that are indicative of the pain by observing the patient, then allocating at least one pain indicator to each feature wherein the indicator is indicative of the severity of the pain and then displaying the indicators simultaneously to provide an assessment of the pain. In a particular embodiment facial features are selected as the indicator.

WO 2017/183994 A1 provides a wearable pain measurement and monitoring device that uses a combination of physiological and behavioural data to assess pain levels in patients. The device is designed to provide accurate and objective pain measurements, which can be useful for pain management and treatment. The device is worn on the wrist and uses a complicated combination of sensors, including an electrodermal activity sensor, a temperature sensor, an accelerometer, and a heart rate monitor, to gather physiological data, such as facial expressions and vocalisations.

WO 2019/204899 A1 is a device that measures pain using thermal imaging technology. It analyses temperature changes in the skin caused by inflammation, which are associated with pain. The device provides a pain score based on the magnitude and duration of these temperature changes. This approach is non-invasive and unlike the present invention does not require physical contact with the patient. In summary, WO 2019/204899 A1 measures pain using thermal imaging technology, while the present invention assesses pain through touch using a haptic device.

The collected date is analysed using machine learning algorithms to identify patterns and correlations that can be used to assess pain levels. The system also includes a user interface that allows patients to self-report pain levels and other relevant information.

Although these techniques are automated and use computers for data storage and assessment display they continue to rely on numerical rating scales associated with the features that are observed to obtain an assessment of the pain. The techniques are therefore an automated method and system which relies on the standard rating scales that have been used for some time despite their shortcomings as has been shown by their over or under estimation of the amount and type of pain.

In addition humans have difficulties in articulating pain and those such as medical practitioners and carers who are responsible for the determination of the pain and the development of appropriate treatment can easily draw inaccurate conclusions and be unable to proscribe the most beneficial treatment.

### SUMMARY

The present invention seeks to overcome these problems and provides a device and an associated method for communicating pain experiences by a multi-sensory arrangement based on a combination of tactile and visual information which can be calibrated to a user to enable personalised pain assessment.

The invention uses touch to provide a quick and intelligent way to access pain providing personalised and more effective pain management. Pain and the sense of touch are both processed in the same part of the human brain known as the somatosensory cortex. When nerves within the body detect these sensations they are passed to the somatosensory cortex.

The invention therefore provides a device comprising pressure sensors that measure the pressure of an applied touch; the duration of the touch; and enables an assessment of pain including the nature of the pain and the magnitude of the pain by analysis of the information gleaned from the touch.

### BRIEF DESCRIPTION OF DRAWINGS

FIG. 1 is an exploded view of a pain assessment device.

FIG. 2 is a schematic illustration of a sensor holder of a pain assessment device.

FIG. 3 is a perspective view of a pain assessment device.

FIG. 4 is a flow chart illustrating information flow during pain assessment with the pain assessment device.

FIG. 5 is a perspective view of a pain assessment device with an outer panel removed.

FIG. 6 is a diagram illustrating various data flows during pain assessment with the pain assessment device.

FIG. 7 is a comparison of pain determination with the pain assessment device and methods of the present teachings with known pain management processes.

### DETAILED DESCRIPTION

The invention therefore provides a device comprising pressure sensors that measure the pressure of an applied touch; the duration of the touch; and enables an assessment of pain including the nature of the pain and the magnitude of the pain by analysis of the information gleaned from the touch.

The device of the present invention comprises a means that can be held in the human hand comprising a first section of a stretchable material of known stiffness whereby a user can transfer their pain tangibly by exerting the associated pressure onto the section. In operation this tangible interface provides information to the other components of the device to enable a picture of the degree and nature of the pain to be developed. The device further comprises a second section, usually a base section, for retention of the various active components of the device. The active components comprise a sensing body stored within the device which comprises a tactile sensor, a micro holder micro controller and a sensor holder these components process the information received from the tangible interface and enable it to be compared with information with previously stored information pertaining to the individual who is being assessed for pain.

A convenient tactile pressure sensor consists of a piezoresistive layer is best sandwiched between two pieces of flexible material such as polyester. Printed conductors preferably silver conductors are on each half. The sensor preferably incorporates a vertical and horizontal array of traces. Such placement can be only applied on a flat surface. The tactile sensor has three important elements; a force sensing resistor, a flexible circuit board and a microcontroller and in a preferred embodiment the force sensing resistor comprise a series of resistors evenly distributed over the surface of the sensor holder.

In a further preferment the force sensing resistors can comprise a series of sensors evenly distributed over the surface of the sensor holder and different sensations originating from the different regions of the touch can be identified, recorded and assimilated. The sensing resistors are preferably linked to a flexible circuit board which in turn is connected to a microcontroller which can send a signal such as via Bluetooth and Wi-Fi to activate and configure a computer to collect and assimilate all the data obtained from the sensors.

Accordingly, the device and method of this invention enable a representation of the pain (however complicated) experienced by an individual such as a patient through the medium of touch. The method captures multiple dimensions of the pain including pain intensity, pain characteristics, the emotional state of the patient and the physical function to which the pain relates as indicated by the reaction to the touch of the patient. If desired these various metrics can be combined into an overall pain score which may then be compared with previous pain scores based on similar metrics to provide an assessment of the progress of the pain over time.

The device therefore provides a soft tangible interface that provides feedback after applying force or touch to the device. This feedback enables the computer to which the information is transmitted to transform the feeling of pain into visible and tangible information indicating the condition of a patient. In particular it enables non-verbal assessment of pain by the visualisation of the nature and degree of pain removing the need for verbal expression of the pain which can be difficult for an individual to articulate.

Accordingly the present invention provides patients and practitioners with immediate feedback via a personalised assessment of pain Doctors can view a complete picture of a patient's pain experience through time, e.g. a patient may have had several distinct episodes of severe pain with deteriorating emotional state. These patterns can provide doctors with an improved picture to intervene and adjust treatment. This proactive and data driven pain management arrives at the right treatment plan more quickly, can reduce the need for repeated clinic visits for unsatisfied patients, leading to better pain outcomes as well as a reduction in consultation requirements and the associated healthcare costs.

This invention uses pressure sensors to measure touch pressure, touch duration and spacing. These are used to calculate pain intensity and emotional state. This data can also provide deformation changes that give insights into the pain characteristics, by measuring surface changing force, shape and texture. Pain intensity, emotional state and physical function metrics may then be combined into a pain score that is easy to track over time.

In order to allow for variation in an individual's pain, this invention allows for different input data sources to be assigned varying weights in the pain score. This personalised approach enables tracking pain intensity, emotional state, and physical function metrics over time and report the difference compared to past scores such as by a percentage. Additionally, the data collected from each metric can be stored in individual datasets, which can be used to build models predicting treatment outcomes and changes in pain behaviour. Pain intensity, emotional state, and physical function metrics can affect pain trajectories and can vary among individuals, this invention can assign varying weights to different input data sources in the pain score. This enables the provision of a personalised pain score to individual patients, taking into consideration their unique pain experiences and needs.

The preferred form of the invention will include two AI applications:
A. An AI model for accurate pain scores, multivariate probabilistic model
B. A time analysis model such as a Bayesian time series analysis to identify patterns in a patient's pain through time which will enable the following to be performed.
   1. Data-driven clustering of patients that have a shared response to particular treatments, enabling personalised care and patient stratification.
   2. Understanding the underlying trends which can help in the early prediction of potential deterioration in patient condition.

Moreover, the multidisciplinary/multisensory facets of this invention can be translated into different tools for various needs.

The invention can provide a rehabilitation and self-management tool for pain and stress: it can also help to track and review users' actions. As the user constantly interacts with the device, they are able to build cognitive awareness of their invisible symptoms increasing treatment adherence and reducing the anxiety surrounding their pain. Furthermore, the system will work as an assistive technology that can help to form a remote co-management relationship between healthcare providers and patients offering supported self-management and guidance.

The device of the present invention may also be used for training for people to understand pain: by stretching, pressure, clicking and dragging the soft surface of the device, users are able to simulate their pain experiences. Haptic feedback, which is essentially in a man-machine interface, allows computer simulations of various tasks to relay realistic, tangible sensations to a user. It allows objects simulated visually to take on actual physical properties. The invention allows the incorporation of tactile sensation into virtual or remote objects, users have the ability to push, pull, feel. By manipulating the form of painful experiences that's being created, the user is manipulating their perception of what is going on their body.

The present invention is illustrated by reference to the accompanying drawings.

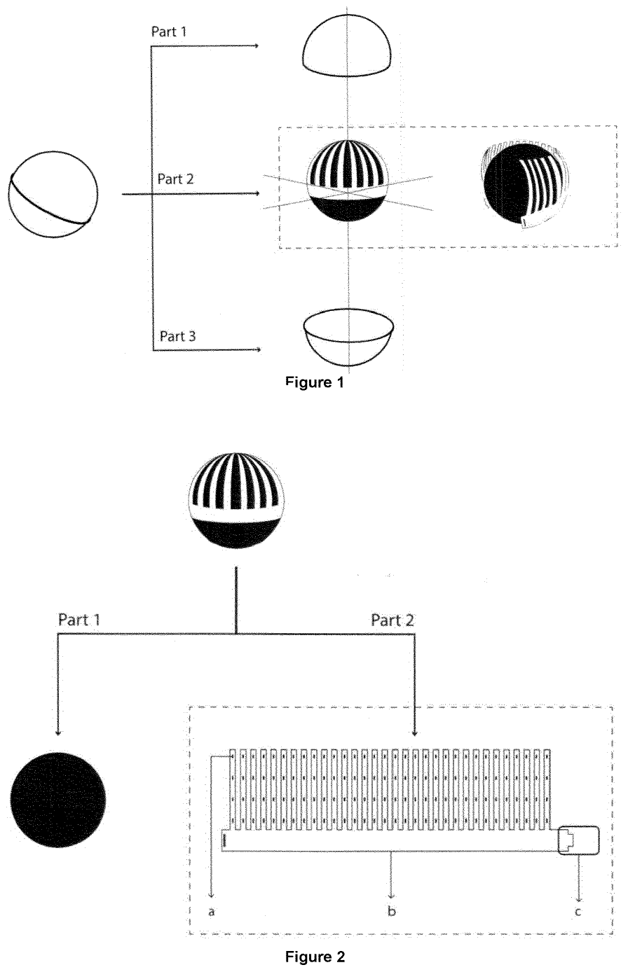

Referring to FIG. 1 Part 1 of the device of the present invention comprises a control panel (1) that is designed to be assembled around the top part (2) of the device which is made of soft stretchable resilient material that can register the pain of users. Part 2 of the device is the sensing component of the device which is stored in the base (3) and comprises a tactile sensor, a microcontroller and a sensor holder as shown in FIG. 2.

The tactile sensor includes a force sensing resistor (4); a flexible circuit board (5) and a microcontroller (6). The forcing sensor resistance of component (4) are distributed over the surface of the sensor holder, the flexible circuit board (5) is shaped so that it can be placed around the top part (2). The microcontroller is connected to the circuit board and is capable of sending signals to data collection by, for example, Bluetooth or Wi-Fi.

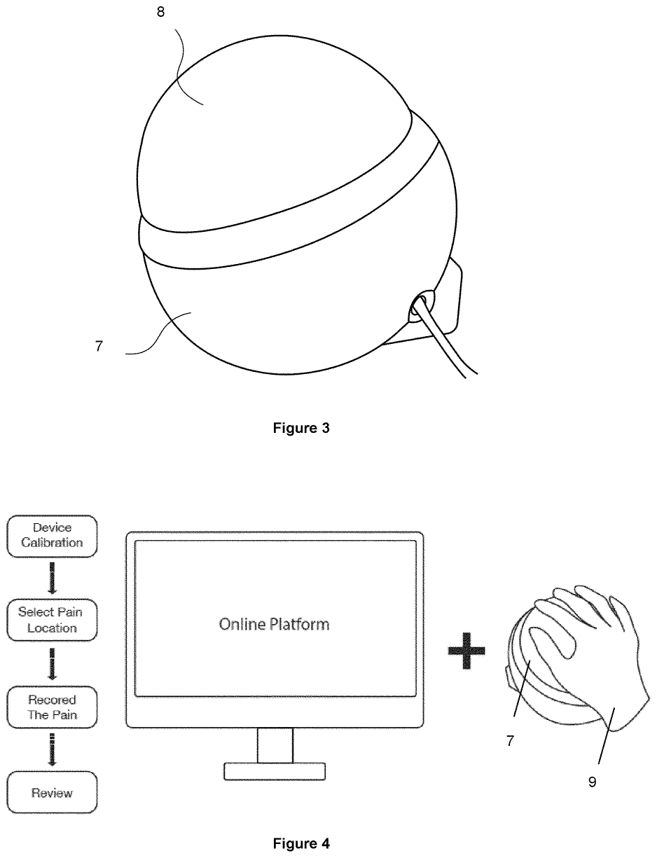

FIG. 3 shows a device according to the invention comprising a base (7) containing the components of the device and an upper panel used (8) for the touch required to operate the device.

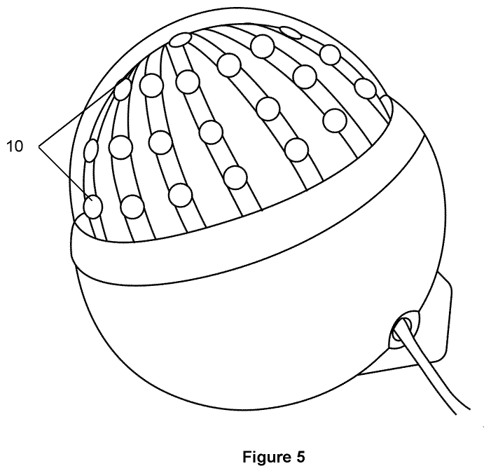

FIG. 4 is a flow chart indicating the information flow during pain assessment showing a hand (9) placed on the upper panel.

FIG. 5 shows the upper touch area with the outer panel removed to show the distribution of the touch sensing elements (10) located beneath the panel.

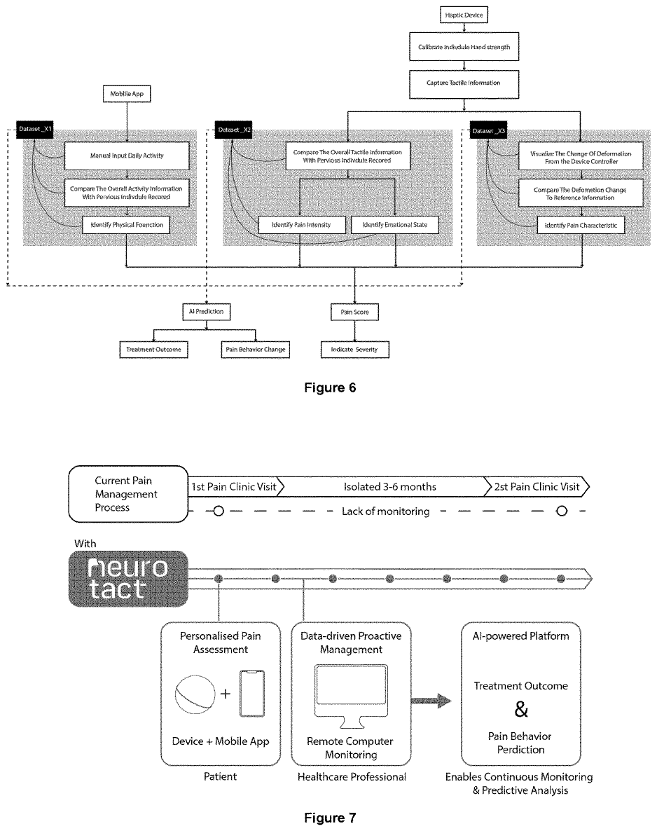

FIG. 6 is a diagram showing the various data flows involved in the pain assessment and FIG. 7 compares the pain determination according to the invention with certain current pain management processes.

What is claimed is:

1. A device comprising:
a plurality of pressure sensors which measure a pressure of an applied touch and a duration of the applied touch; and
wherein the plurality of pressure sensors enable an assessment of a pain pertaining to an individual including a nature of the pain and a magnitude of the pain.

2. The device according to claim 1, comprising:
a means that can be held in a human hand comprising a first section of a stretchable material of known stiffness; and
whereby the individual can transfer their pain tangibly by exerting a pressure associated with their pain onto the first section.

3. The device according to claim 2, wherein the means is a tangible interface which provides information to other components of the device to enable a picture of a degree and the nature of the pain to be developed.

4. The device according to claim 3, wherein the device includes a second section for retention of a plurality of active components of the device.

5. The device according to claim 4, wherein the plurality of active components comprise:
i) a sensing body stored within the device which comprises a tactile sensor;
ii) a microcontroller; and
iii) a sensor holder, and
wherein the plurality of active components process the information received from the tangible interface and enable the information to be compared with previously stored information pertaining to the individual who is being assessed for the pain.

6. The device according to claim 1, wherein the plurality of pressure sensors include a plurality of tactile sensors which have three elements;
i) a plurality of force sensing resistors;
ii) a flexible circuit board; and
iii) a microcontroller.

7. The device according to claim 6 wherein the plurality of force sensing resistors comprise a series of resistors evenly distributed over a surface of a sensor holder.

8. The device according to claim 6, wherein the plurality of force sensing resistors comprise a series of force sensing resistors evenly distributed over a surface of a sensor holder and different sensations originating from different regions of the applied touch can be identified, recorded, and assimilated.

9. The device according to claim 6, wherein the plurality of force sensing resistors are linked to the flexible circuit board;
wherein the flexible circuit board is connected to the microcontroller; and
wherein the microcontroller is configured to send a wireless signal to activate and configure a computer to collect and assimilate all the data obtained from the plurality of pressure sensors.

10. A method for assessment of the pain comprising using the device according to claim 1 to capture multiple dimensions of the pain including a pain intensity, one or more pain characteristics, an emotional state of a patient which is the individual, and a physical function to which the pain relates.

11. The method according to claim 10 in which a plurality of metrics are combined into an overall pain score.

12. The method according to claim 11 in which the overall pain score is compared with previous pain scores based on similar metrics.

13. The device according to claim 9, wherein the wireless signal is Bluetooth or Wi-Fi.
